(19) United States
(12) Patent Application Publication
(10) Pub. No.: US 2025/0180762 A1
(43) Pub. Date: Jun. 5, 2025

(54) COOLING APPARATUS FOR DISSIPATING HEAT FROM AN X-RAY CONVERTER ELEMENT, CT DETECTOR MODULE AND CT DEVICE

(71) Applicant: Siemens Healthineers AG, Forchheim (DE)

(72) Inventors: Lukas RUHMANN, Gremsdorf (DE); Michael TEUBER, Forchheim (DE); Gottfried TSCHOEPA, Baiersdorf (DE); Stefan WOELFEL, Dormitz (DE); Oleksandr MAKSYMENKO, Zwickau (DE)

(73) Assignee: Siemens Healthineers AG, Forchheim (DE)

(21) Appl. No.: 18/960,363

(22) Filed: Nov. 26, 2024

(30) Foreign Application Priority Data
Nov. 30, 2023 (DE) 10 2023 211 984.6

(51) Int. Cl. G01T 1/24; A61B 6/00; A61B 6/03; G01T 1/20

(52) U.S. Cl. CPC G01T 1/244; G01T 1/20188; A61B 6/035; A61B 6/4488

(57) ABSTRACT

A cooling apparatus comprises a plurality of cooling elements arranged in a row along a first direction. Each cooling element has a metallic material that has a plurality of continuous tunnel-shaped cavities along the first direction. The cavities are configured for through-flow by a fluid along the first direction. The cooling elements are configured to make surface-to-surface contact with a corresponding thermal contact surface of the X-ray converter element, and the cooling elements are arranged with respect to each other in the row such that the respective cavities overlap at least partially at interfaces between adjacent cooling elements.

### CROSS-REFERENCE TO RELATED APPLICATION(S)

The present application claims priority under 35 U.S.C. § 119 to German Patent Application No. 10 2023 211 984.6, filed Nov. 30, 2023, the entire contents of which is incorporated herein by reference.

### FIELD

The present invention relates to a cooling apparatus for dissipating heat from an X-ray converter element, to a CT detector module and to a CT device.

### BACKGROUND

Modern computed tomography devices (CT devices) have a gantry with a rotatable frame, on which are mounted, inter alia, the X-ray source and a detector module for detecting the X-ray radiation. Such a CT detector module normally comprises an X-ray converter element, which has an X-ray sensor layer and, if applicable, an underlying layer containing A/D (analog-to-digital) converters. Electronic integration has recently been an important trend in X-ray converter elements. The aim of this integration has been to reduce the length of the analog path between the analog X-ray sensor layer and the A/D converters, which are usually realized as an application-specific integrated circuit (ASIC). In the case of integrating X-ray converters, the analog X-ray sensor layer is formed by a suitable sensor layer, for instance a scintillator, in combination with a photodiode, whereas counting X-ray converters use a direct conversion semiconductor sensor. An A/D converter in the form of an ASIC then generates the digital output signal. In both cases, integration of the ASICS into a compact structure, in particular a stacked structure, together with the analog X-ray sensor layer brings a major heat source closer to the X-ray sensor elements of the X-ray sensor layer itself.

Since the X-ray sensor elements react highly sensitively to heat variations, thermal management is a crucial function in the development of a modern CT detector module. The particular challenges of thermal management in this context are keeping the operating temperature of the CT detector module stable, avoiding temperature gradients between adjacent X-ray converter elements, and also reducing the temperature gradients within each X-ray converter element. These challenges are even more significant in counting X-ray converters, because the direct conversion semiconductor sensors are additional sources of heat while also having a sensor performance that reacts highly sensitively to thermal variations.

Thermal management is currently guaranteed by producing a thermal interface between the X-ray converter element and the metal frame of the CT detector module through a thermal adhesive or a thermal paste. This does not allow the transport of a defined amount of heat, however, because the quality of the thermal interface depends on various factors that are not easy to control, in particular on the thickness of the gap between the X-ray converter and the metal frame, and on the precise distribution of the thermal paste.

As a result of the large amount of data from modern CT (counting) detectors, there is increasing demand, because of the high powers of the ASICs, for efficient cooling of the modules in order to keep the sensors made of semiconductor material in the ideal operating range in terms of their temperature. In addition to the absolute temperature, the temperature distribution over the sensor surface plays a crucial role here. Normally, the sensor elements that are closer to the apertures for the cooling-air intake have a lower temperature, because here most of the incoming cooling air is still present, and the cooling fins are exposed to a frontal flow. In addition, air is thereby displaced into the rear area of the detector, with the sensors in the region furthest away from the intake aperture thus receiving less cooling air, which moreover is additionally heated by the remaining sensor boards.

### SUMMARY

An object of one or more embodiments of the present invention is to facilitate more efficient heat dissipation for a CT detector module.

At least this object is achieved, according to one or more embodiments of the present invention, by the subject matter of the independent claims. The subject matter of the dependent claims contains advantageous embodiments including expedient developments.

Independent of the grammatical term usage, individuals with male, female or other gender identities are included within the term.

In a first aspect, an embodiment of the present invention relates to a cooling apparatus for dissipating heat from an X-ray converter element. The cooling apparatus comprises a plurality of cooling elements. The cooling elements are arranged in a row along a first direction. The cooling elements each have a metallic material that has a plurality of continuous tunnel-shaped cavities along the first direction, which are designed for the through-flow by a fluid along the first direction. The cooling elements are designed to make surface-to-surface contact with a corresponding thermal contact surface of the X-ray converter element. The cooling elements are arranged with respect to each other in the row arrangement in such a way that the respective cavities overlap at least partially at interfaces between adjacent cooling elements.

The cooling apparatus advantageously comprises a plurality of cooling elements that can be identical or different in design. Each of the cooling elements can have a metallic material, in particular be formed largely from a metallic material. The cooling elements can advantageously have a metallic material of high thermal conductivity. In addition, the plurality of cooling elements can have the same or at least partially different metallic materials. For example, the metallic material can comprise a substrate, an extruded material and/or a cast material. Moreover, the metallic material can comprise an aluminum alloy and/or copper.

The metallic material of the cooling elements can have a plurality of continuous tunnel-shaped cavities along the first direction. The cavities of each one of the cooling elements respectively can be identical or differ in design at least in some respects. For example, the cavities can differ in their geometric shape and/or extent and/or in their spatial course and/or their spatial orientation.

The first direction can denote a first spatial direction. The plurality of cooling elements are arranged in a row along the first direction. Each of the cooling elements can be adjacent to at least one further cooling element respectively along the first direction, in particular can abut the at least one further cooling element or be spaced apart from the at least one further cooling element. It is thereby possible to form the row arrangement of the plurality of cooling elements along the first direction.

The cavities can each be tunnel-shaped, in particular in the form of a pipe and/or tube, and/or be cylindrical and/or cuboid in shape. The cavities can be surrounded by the metallic material, in particular bounded by the metallic material. In particular, the cavities can each be surrounded by the material along the directions that differ from the first direction. Advantageously, the cavities can each run from one end of the material along the first direction continuously to an opposite end of the material, in particular in a straight or curved line. The plurality of cavities can be separate, in particular fluid-tight, with respect to each other, or at least partially connected.

The cavities are each designed for the through-flow by the fluid, for example a liquid, in particular water, and/or a gas, in particular air, along the first direction, in particular from the one end of the material along the first direction to the opposite end of the material. In particular, the cavities can be designed to conduct, in particular transport, the fluid along the first direction. Advantageously, the cavities, in particular a longitudinal direction and/or an axis of symmetry of the cavities, can run substantially parallel to each other. In addition, the cavities, in particular the longitudinal direction and/or the axis of symmetry of the cavities, can run substantially parallel to the first direction.

The cooling elements, in particular an external face of the respective cooling element, can be designed to make surface-to-surface contact, in particular, contact in the manner of a positive fit, with a corresponding thermal contact surface of the X-ray converter element. The external face of the respective cooling element can be an external interface, in particular surface, of the metallic material. Advantageously, the plurality of cooling elements can be designed to make surface-to-surface contact in each case with a corresponding thermal contact surface of the X-ray converter element in the row arrangement of the plurality of cooling elements. Advantageously, the cooling elements can each make surface-to-surface contact with the corresponding thermal contact surface in such a way that heat can be exchanged, in particular transferred, between the X-ray converter element and the cooling elements.

Advantageously, the cooling elements are arranged with respect to each other in the row arrangement in such a way that the respective cavities overlap at least partially, in particular entirely, at the interfaces between adjacent cooling elements. Advantageously, the cavities can overlap at least partially at the interfaces in such a way that the fluid can flow along the first direction from the cavities of one cooling element into the cavities of the respectively adjacent cooling element.

By arranging, in particular mounting, the cooling elements on the thermal contact surface of the CT detector module, a gap between sensor board and cooling element can be kept as small as possible. Since these cooling elements can be manufactured separately, in particular independently, of a module carrier, a multiplicity of manufacturing methods are possible here. This advantageously allows materials of higher thermal conductivity to be chosen.

The proposed embodiment can facilitate efficient heat dissipation for a CT detector module.

In a further advantageous embodiment of the proposed cooling apparatus, the cooling elements can each have on an external face, away from the first direction, a recess and/or protrusion designed to make surface-to-surface contact with the corresponding thermal contact surface of the X-ray converter element.

Advantageously, the cooling elements each have a recess and/or protrusion on an external face, the surface of which has a normal vector running non-parallel, in particular perpendicular, to the first direction. The external face of the respective cooling elements can be an external interface, in particular surface, of the metallic material. The recess can comprise in particular a groove and/or step and/or collar. In addition, the protrusion can comprise a spring and/or rod and/or pin and/or platform. The recess and/or protrusion can be designed to make contact in the manner of a positive fit with a corresponding thermal contact surface of the X-ray converter element. Advantageously, the plurality of cooling elements can each be designed to have a recess and/or protrusion that make contact in the manner of a positive fit in each case with a corresponding thermal contact surface of the X-ray converter element in the row arrangement of the plurality of cooling elements. Advantageously, the recesses and/or protrusions of the cooling elements can each make contact in the manner of a positive fit with the corresponding thermal contact surface in such a way that heat can be exchanged, in particular transferred, between the X-ray converter element and the cooling elements. In particular, by virtue of the contact made by the corresponding thermal contact surface with the recess and/or protrusion of the cooling element, in particular with the metallic material, thermal contact can be made by metal-to-metal contacts that therefore have a high thermal conductivity.

The proposed embodiment can facilitate improved thermal contact to be made with the X-ray converter element by the cooling elements of the cooling apparatus for dissipating heat.

In a further advantageous embodiment of the proposed cooling apparatus, at least one of the cooling elements can comprise a plurality of cooling fins as part of the material, which are mutually spaced in a stacked arrangement. A stacking direction of the cooling fins can differ from the first direction, and voids formed between the cooling fins can form the cavities.

The at least one cooling element can comprise a frame, which is designed to hold the plurality of cooling fins in the mutually spaced and stacked arrangement. Advantageously, the frame and the plurality of cooling fins can comprise the metallic material, in particular be formed from the metallic material. The cooling fins can be in thermal contact with the frame. In addition, the recess and/or protrusion can be arranged on an external face of the frame.

The plurality of cooling fins can be spaced equidistantly or at different distances from one another. Advantageously, the plurality of cooling fins can be arranged in a stack parallel to one another, in particular parallel to the first direction. In addition, the cooling fins can be substantially flat, in particular planar. The cooling fins can be stacked in such a way that the flat faces of adjacent cooling fins face one another. The stacking direction of the cooling fins can differ from the first direction, in particular can have a specified angle with respect to the first direction. Voids formed between the cooling fins, which voids are bounded in particular by the shared frame, can form the cavities. The cooling fins and the frame can be designed as a single piece, in particular have an integral design, or as multiple pieces. In particular, the cooling fins and one or more frame pieces can be adhesively bonded to form the associated cooling element.

The proposed embodiment can facilitate efficient provision of the cooling elements for the cooling apparatus according to one or more embodiments of the present invention. In particular, a significant increase in the number of cooling fins in the cooling element can increase a cooling capacity significantly compared with a conventional cooling element.

In a further advantageous embodiment of the proposed cooling apparatus, the stacking direction can run perpendicular to the first direction.

Advantageously, the stacking direction can run perpendicular, in particular at an angle of 90 degrees, to the first direction. This can advantageously ensure that the cavities formed by the voids are designed for the through-flow by the fluid along the first direction.

The proposed embodiment can allow the fluid to flow around the cooling fins, in particular without obstruction, along the first direction.

In a further advantageous embodiment of the proposed cooling apparatus, the cavities can be arranged equidistantly perpendicular to the first direction in a cross-sectional surface of the associated cooling element.

Advantageously, the cavities can be arranged equidistantly from one another perpendicular to the first direction in at least one cross-sectional surface of the associated cooling element. In particular, the cavities can be arranged equidistantly from one another in all cross-sectional surfaces over an entire length of the associated cooling element along the first direction, in particular can additionally run parallel to one another.

The proposed embodiment can allow a uniform fluid distribution within the cooling elements during the through-flow of the cavities.

In a further advantageous embodiment of the proposed cooling apparatus, the cavities can be spaced at different distances from one another perpendicular to the first direction in a first cross-sectional surface of the associated cooling element.

Advantageously, the cavities can be spaced at different distances from one another perpendicular to the first direction in at least one cross-sectional surface of the associated cooling element. In particular, the cavities can be spaced at different distances from one another in all cross-sectional surfaces over an entire length of the associated cooling element along the first direction, in particular can additionally run parallel to one another.

The proposed embodiment can allow an irregular arrangement of the cavities for improved heat dissipation to the fluid flowing through, for instance by an optimized arrangement of the cavities in terms of the thermal contact surface.

In a further advantageous embodiment of the proposed cooling apparatus, at least one of the cooling elements can be designed as a single piece.

Fabricating at least one of the cooling elements, in particular each of the cooling elements, as a single piece, in particular integrally, can advantageously avoid construction costs during production of the cooling element.

For example, this allows savings on material and fastenings and on time-intensive processing steps. In addition, when in the form of a single piece, the at least one cooling element can advantageously be formed entirely from the metallic material. This means that the at least one cooling element can have a homogeneous thermal conductivity.

In a further advantageous embodiment of the proposed cooling apparatus, the at least one single-piece cooling element can be produced via an additive manufacturing technique.

An additive manufacturing technique comprises a process in which a component is constructed by material deposition layer by layer on the basis of digital 3D construction data. This allows a high degree of variability in the design. Advantageously, this even allows structures and forms that are complex yet stable to be produced efficiently in terms of both time and resources. Depending on the starting material and usage, the components can be manufactured using a method of stereolithography, laser sintering or 3D printing. Various metals, plastics and composites are available as the materials. In particular, the metallic material can exist as a powdered substrate at the start of the additive manufacture of the at least one cooling element.

For example, the at least one cooling element can be produced via selective laser melting (SIM) or selective laser sintering (SLS). A powdered material comprising a metal powder is preferably used in this case. First, a thin layer of the powdered material is applied to a build platform. Via a laser, the powder can be melted precisely at the locations specified by the computer-generated component construction data. The manufacturing platform is then lowered and a further application of power is made. The material is melted again, and fuses with the underlying layer at the defined locations.

Advantageously, an additive manufacturing technique, and especially advantageously in particular a method of selective laser melting or selective laser sintering, can achieve a high bulk density of the metallic material in the at least one cooling element. In addition, the at least one cooling element can be constructed more quickly via an additive manufacturing technique, or less expensive materials can be used.

In a further advantageous embodiment of the proposed cooling apparatus, the at least one single-piece cooling element can be produced via a subtractive manufacturing technique.

A subtractive manufacturing technique comprises a process in which a component is produced by material removal on the basis of digital 3D construction data. This allows a high degree of variability in the design. Depending on the initial material and usage, the components can be manufactured using a method of turning, grinding, drilling, milling and/or cutting. Various metals, plastics and composites are available as the materials.

The material can preferably be removed at the locations specified by the computer-generated component construction data. Various removal techniques can be applied individually, sequentially or in combination, for instance turning, grinding, drilling, milling and/or cutting.

Advantageously, this even allows structures and forms that are complex yet stable to be produced efficiently in terms of both time and resources.

In a further advantageous embodiment of the proposed cooling apparatus, different ones of the plurality of cooling elements in the row arrangement can have a different cavity volume and/or a different cavity density and/or a different number of cavities and/or a different cavity surface.

Advantageously, the cooling elements along the first direction, in particular along the direction of the row arrangement of the plurality of cooling elements, can have an at least partially, in particular entirely different number and/or cavity density, in particular density of cavities, and/or can have a different cavity volume, in particular volume of the cavities, and/or a different cavity surface. The number of cavities can be defined here as the number of related cavities in one of the respective cooling elements. Alternatively or additionally, the number of cavities can be defined as the number of inlets and outlets, in particular apertures, of the cavities at opposite ends of the respective cooling element along the first direction. The density of the cavities can be defined as the ratio of a volume of the cavities to a volume of the, in particular metallic, material of the respective cooling element. Alternatively or additionally, the cooling elements can have a different volume of the cavities along the first direction, in particular for a constant density of the cavities. Alternatively or additionally, the cooling elements can have a different cavity surface along the first direction. The cavity surface can denote a surface of the material at the interfaces with the cavities of one cooling element. The interfaces between the cavities and the material for the particular cooling element can form a thermal contact surface between the fluid flowing through and the metallic material.

The heat dissipation capacity of the cooling apparatus can advantageously be adjustable along the first direction by the proposed embodiment. In particular, the proposed embodiment makes it possible that a surface of the cavities that is used for convection can be matched precisely to dissipated power to be carried away, and also to the amount of fluid flowing through the particular cooling element and to the fluid temperature of the shared fluid flow.

In a further advantageous embodiment of the proposed cooling apparatus, the different ones of the plurality of cooling elements can have in the row arrangement parallel to the first direction a decreasing cavity volume and/or a decreasing cavity density and/or an increasing number of cavities and/or an increasing cavity surface.

Advantageously, the cooling elements in the row arrangement, in particular along the first direction, can have a, in particular monotonically, decreasing, in particular reducing, cavity volume parallel to the first direction. Hence the plurality of cooling elements can each have a smaller cavity volume compared with the respectively preceding cooling element parallel to the first direction. Alternatively or additionally, the cooling elements in the row arrangement, in particular along the first direction, can have a, in particular monotonically, decreasing, in particular reducing, cavity density parallel to the first direction. Hence the plurality of cooling elements can each have a lower cavity density compared with the respectively preceding cooling element parallel to the first direction. Alternatively or additionally, the cooling elements in the row arrangement, in particular along the first direction, can have an, in particular monotonically, increasing, in particular rising, cavity surface parallel to the first direction. Hence the plurality of cooling elements can each have a larger cavity surface compared with the respectively preceding cooling element parallel to the first direction.

The heat dissipation capacity of the cooling apparatus can advantageously be maintained along the first direction by the proposed embodiment. The higher a fluid temperature and the lower an amount of fluid flowing through, the larger a surface of the respective cavities must be in order to be able to release the same amount of heat to the fluid. Advantageously, the proposed embodiment can effectively prevent a temperature gradient forming in the X-ray converter element along the first direction, in particular along the direction of through-flow by the fluid.

In a further advantageous embodiment of the proposed cooling apparatus, the cooling elements can have in the row arrangement parallel to the first direction an increasing number of cooling fins.

Advantageously, the plurality of cooling elements can each have a larger number of cooling fins compared with the respectively preceding cooling element parallel to the first direction. This can advantageously realize an increase in cavity surface along the first direction.

In a further advantageous embodiment of the proposed cooling apparatus, the cooling elements can have, away from the first direction, a closed profile, so that the cavities in the row arrangement of the plurality of cooling elements form a closed cooling duct.

The cavities can each be tunnel-shaped, in particular in the form of a pipe and/or tube, and/or be cylindrical and/or cuboid in shape. The cavities can be surrounded by the metallic material, in particular bounded by the metallic material, away from the first direction. In particular, the cavities can each be surrounded by the material along the directions that differ from the first direction. Advantageously, the cavities can each run from one end of the material along the first direction continuously to an opposite end of the material, in particular in a straight or curved line. This advantageously allows a design of the cooling elements as self-contained cooling elements containing the plurality of cavities. By virtue of the closed profile of the individual cooling elements and the row arrangement of the plurality of cooling elements, a closed cooling duct can advantageously be formed along the first direction that prevents the fluid escaping into a rear area of a CT detector. In a construction having a plurality of X-ray converter elements, a sufficient, in particular full, fluid flow is thereby achieved even for the X-ray converter elements that are arranged furthest from an entry aperture for the fluid along the first direction, and an available cooling capacity of the fluid can be used efficiently.

In a second aspect, an embodiment of the present invention relates to a CT detector module. The CT detector module comprises at least one X-ray converter element and a cooling apparatus according to an embodiment of the present invention. The X-ray converter element has a top face and a bottom face. The X-ray converter element has on its top face an X-ray detector layer, and on its bottom face at least one thermal contact surface. In an operating state of the CT detector module, the X-ray converter element is in heat-conducting contact with the cooling apparatus via the thermal contact surface.

The advantages of the proposed CT detector module are essentially the same as the advantages of the proposed cooling apparatus. Features, advantages or alternative embodiments mentioned in this connection can also be applied to the other claimed subject matter, and vice versa.

The X-ray converter element has on its top face an X-ray detector layer. In an operating state of the CT detector module, this can face the X-ray source. In addition, the X-ray detector layer can be designed to detect X-rays emitted by the X-ray source. The X-ray detector layer can comprise a direct conversion (semiconductor) X-ray sensor layer, for example having CdTe, CdZnTe, CdTeSe, CdZnTeSe, CdMnTe, GaAs, Si or Ge as the semiconductor material. The X-ray detector layer can also comprise an X-ray sensor layer which is designed to convert X-ray radiation into light and optically coupled photodiodes, in particular one or more photodiode arrays. Scintillator material, for instance GOS (Gd2O2S), CsJ, YGO or LuTAG, is often used here as the material. The X-ray detector layer can also comprise a layer containing analog-to-digital converters, onto which the X-ray sensor layer is applied, where the A/D converter layer can be realized in one or more ASICs. The X-ray detector layer can be applied to a base board, also called material, for instance a printed circuit board or a ceramic or glass material, which then forms the bottom face of the X-ray converter element.

The X-ray converter element has on its bottom face a, in particular metallic, thermal contact surface, which, in the operating state of the CT detector module, is in heat-conducting, in particular thermal, contact with the cooling apparatus. The heat-conducting contact can be improved further, for example, by thermal paste or thermal pads or adhesives or solder materials. The plurality of cooling elements of the cooling apparatus can be arranged, in the operating state, in a row on the bottom face of the X-ray converter element in such a way that the recess and/or protrusion of each of the cooling elements is in heat-conducting contact with the corresponding thermal contact surface of the X-ray converter element. For this purpose, the cooling elements can be arranged, for example, in a shared module carrier, which allows the cooling elements to be arranged in a row and has a recess at the at least one thermal contact surface. The at least one thermal contact surface can have a metal covering, for example. For instance, the metallic covering can be formed by coating with a metal that is a good thermal conductor, for example gold, silver or copper. The metallic covering can be what is known as a metalized thermal pad. The at least one thermal contact surface can cover part, or even all, of the bottom face of the X-ray converter element. The thermal contact surface can be connected by metalized vias in a base board and/or in the A/D converter layer of the X-ray detector layer to the A/D converter layer and/or the X-ray detector layer so that the heat can be conducted away from there into the thermal contact surface. This is also referred to as thermal via technology. For example, the metalized vias can be contact holes in the base board.

Advantageously, in the operating state of the CT detector module, the fluid, for example cooling air, can flow through the cooling apparatus along the first direction. The fluid, in particular the cooling air, can be provided by a central fluid-provider element, for example a central fan, for a plurality of CT detector modules. Alternatively, the CT detector module can have a dedicated fluid-provider element, for example a dedicated fan, which is designed to provide the fluid in the operating state. For example, the CT detector module can have a fluid duct, in particular an air duct. The cooling apparatus, in particular the plurality of cooling elements, can be arranged in the fluid duct, in particular along the fluid duct. In addition, the dedicated fluid-provider element can be arranged at an aperture of the fluid duct and, in the operating state of the CT detector module, provide a fluid flow in the fluid duct, in particular in the cavities of the cooling apparatus.

In a further advantageous embodiment of the proposed CT detector module, in an operating state of the CT detector module, the X-ray converter element can be in heat-conducting contact with each of the cooling elements of the cooling apparatus via a corresponding thermal contact surface in each case.

The X-ray converter element advantageously has on its bottom face for each of the cooling elements a corresponding, in particular metallic, thermal contact surface, which in the operating state of the CT detector module is in heat-conducting, in particular thermal, contact with the cooling elements of the cooling apparatus.

This can facilitate improved heat dissipation from the X-ray converter element, in particular apportioned to the plurality of cooling elements.

In a further advantageous embodiment of the proposed CT detector module, in an operating state of the CT detector module, the X-ray converter element can be in heat-conducting contact with each of the cooling elements of the cooling apparatus via a corresponding thermal contact surface by a thermally conductive medium. In addition, the thermally conductive media can have different thermal conductivities.

The heat-conducting contact between the thermal contact surfaces and the cooling elements can be provided by a thermally conductive medium in each case. For example, the thermally conductive media can comprise thermal pastes, thermal pads, adhesives and/or solder materials. Advantageously, the thermally conductive media can have different thermal conductivities. In particular, the thermally conductive media can have an increasing thermal conductivity along the first direction.

Advantageously, the proposed embodiment can effectively prevent a temperature gradient forming in the X-ray converter element along the first direction, in particular along the direction of through-flow by the fluid.

In a third aspect, an embodiment of the present invention relates to a CT device comprising at least one CT detector module according to an embodiment of the present invention.

The advantages of the proposed CT device are essentially the same as the advantages of the proposed CT detector module. Features, advantages or alternative embodiments mentioned in this connection can also be applied to the other claimed subject matter, and vice versa.

The CT device can comprise an X-ray source, a proposed CT detector module and a provider unit. The X-ray source and the CT detector module can be arranged opposite one another. The X-ray source can be designed to expose the CT detector module to X-ray radiation along a direction of incidence of the X-rays. The CT device can also comprise a gantry having a rotor. The X-ray source and the CT detector module can be located on the rotor in a defined arrangement, in particular integrated in the rotor or attached to the rotor. The rotor can be mounted to rotate about a rotational axis. An object under examination to be imaged, for example a human and/or animal patient and/or an examination phantom, can be positioned on a patient couch and be movable along the rotational axis through the gantry.

### BRIEF DESCRIPTION OF THE DRAWINGS

Example embodiments of the present invention are described in more detail below and illustrated in the drawings. The same reference signs are used for the same features in different figures, in which:

FIG. 1 shows a schematic representation of an advantageous embodiment of a plurality of cooling elements;

FIG. 2 shows a schematic representation of an advantageous embodiment of a proposed cooling apparatus;

FIG. 3 and FIG. 4 show schematic representations of various advantageous embodiments of a cooling element;

FIG. 5 shows a schematic representation of an advantageous embodiment of a proposed CT detector module;

FIG. 6 shows a schematic representation of a CT device.

### DETAILED DESCRIPTION

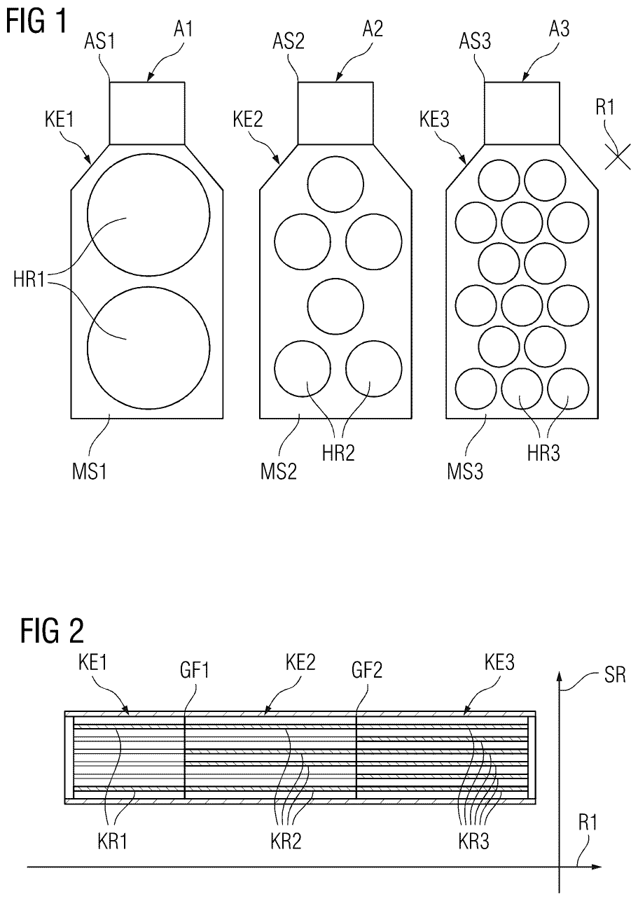

FIG. 1 shows a schematic representation of an advantageous embodiment of a plurality of cooling elements KE1, . . . , KE3. The cooling elements KE1, . . . , KE3 can be arranged in a row along a first direction (shown here side by side). In addition, the cooling elements KE1, . . . , KE3 can each have a metallic material MS1, . . . , MS3 that has a plurality of continuous tunnel-shaped cavities HR1, . . . , HR3 along the first direction R1. The cavities HR1, . . . , HR3 can be designed for the through-flow by a fluid along the first direction R1. The cooling elements KE1, . . . , KE3 can be designed to make surface-to-surface contact with a corresponding thermal contact surface (not shown here) of an X-ray converter element. In particular, the cooling elements KE1, . . . , KE can each have on an external face A1, . . . , A3, away from the first direction R1, a protrusion AS1, . . . , AS3, which is designed to make contact in the manner of a positive fit with a corresponding thermal contact surface of an X-ray converter element. The cavities HR1, . . . , HR3 can be spaced at different distances from one another perpendicular to the first direction R1 in a cross-sectional surface of the associated cooling element KE1, . . . , KE3. Advantageously, at least one of the cooling elements KE1, . . . , KE3 can be designed as a single piece. In particular, the at least one single-piece cooling element KE1, . . . , KE3 can be produced via an additive manufacturing technique. Alternatively, the at least one single-piece cooling element KE1, . . . , KE3 can be produced via a subtractive manufacturing technique.

FIG. 2 shows a schematic representation of an advantageous embodiment of a proposed cooling apparatus. The cooling elements KE1, . . . , KE3 can be arranged in a row along the first direction R1 in such a way that the respective cavities HR1, . . . , HR3 overlap at least partially at the interfaces GF1, GF2 between adjacent cooling elements KE1, . . . , KE3. The cavities HR1, . . . , HR3 of each one of the cooling elements KE1, . . . , KE3 respectively can run substantially parallel to one another.

Advantageously, different ones of the plurality of cooling elements KE1, . . . , KE3 in the row arrangement can have a different cavity volume HR1, . . . , HR3 and/or a different cavity density HR1, . . . , HR3 and/or a different number of cavities HR1, . . . , HR3 and/or a different cavity surface. In particular, the different ones of the plurality of cooling elements KE1, . . . , KE3 can have in the row arrangement parallel to the first direction R1 a decreasing cavity volume and/or a decreasing cavity density and/or an increasing number of cavities HR1, . . . , HR3 and/or an increasing cavity surface. In addition, the cooling elements KE1, . . . , KE3 can comprise a plurality of cooling fins KR1, . . . , KR3 as part of the material, which are mutually spaced in a stacked arrangement. Moreover, a stacking direction SR of the cooling fins KR1, . . . , KR3 can differ from the first direction R1, and voids formed between the cooling fins KR1, . . . , KR3 can form the cavities HR1, . . . , HR3. Advantageously, the cooling elements KE1, . . . , KE3 can have in the row arrangement parallel to the first direction R1 an increasing number of cooling fins KR1, . . . , KR3. In addition, the cavities HR1, . . . , HR3 can be arranged equidistantly perpendicular to the first direction R1 in a cross-sectional surface of the associated cooling element KE1, . . . , KE3.

Moreover, the cooling elements KE1, . . . , KE3 can have, away from the first direction R1, a closed profile, so that the cavities HR1, . . . , HR3 in the row arrangement of the plurality of cooling elements KE1, . . . , KE3 form a closed cooling duct along the first direction R1.

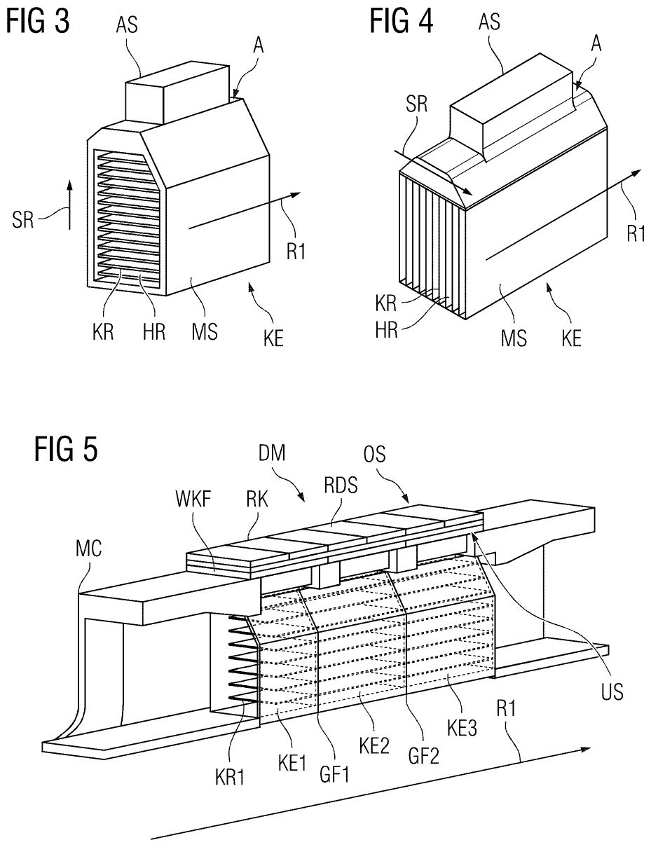

FIGS. 3 and 4 show schematic representations of various advantageous embodiments of a cooling element KE by way of example. The cooling element KE can comprise a plurality of cooling fins KR, which are mutually spaced in a stacked arrangement inside the metallic material MS. The cooling element KE can have on its external face A perpendicular to the first direction R1 a protrusion AS, which is designed to make contact in the manner of a positive fit with a corresponding thermal contact surface of an X-ray converter element. Voids formed between the cooling fins KR can form the cavities HR. The stacking direction SR of the plurality of cooling fins KR can run perpendicular to the first direction R1. In the embodiment shown in FIG. 3, the stacking direction SR can run vertically, and, in the embodiment shown in FIG. 4, can run horizontally.

FIG. 5 shows a schematic representation of an advantageous embodiment of a proposed CT detector module DM. The CT detector module DM can comprise at least one X-ray converter element RK and a proposed cooling apparatus. The X-ray converter element RK can have a top face OS and a bottom face US. The X-ray converter element RK can have on its top face OS an X-ray detector layer RDS, and on its bottom face US a thermal contact surface WKF. In addition, in an operating state of the CT detector module DM, the X-ray converter element RK can be in heat-conducting contact with the cooling apparatus via the thermal contact surface WKF. For this purpose, the cooling elements KE1, . . . , KE3 can be arranged, for example, in a shared module carrier MC, which allows the cooling elements to be arranged in a row and has a recess at the at least one thermal contact surface.

Advantageously, in an operating state of the CT detector module DM, the X-ray converter element RK can be in heat-conducting contact with each of the cooling elements KE, KE1, KE2, KE3 of the cooling apparatus via a corresponding thermal contact surface WKF, in particular by a thermally conductive medium. The thermally conductive media can have different thermal conductivities.

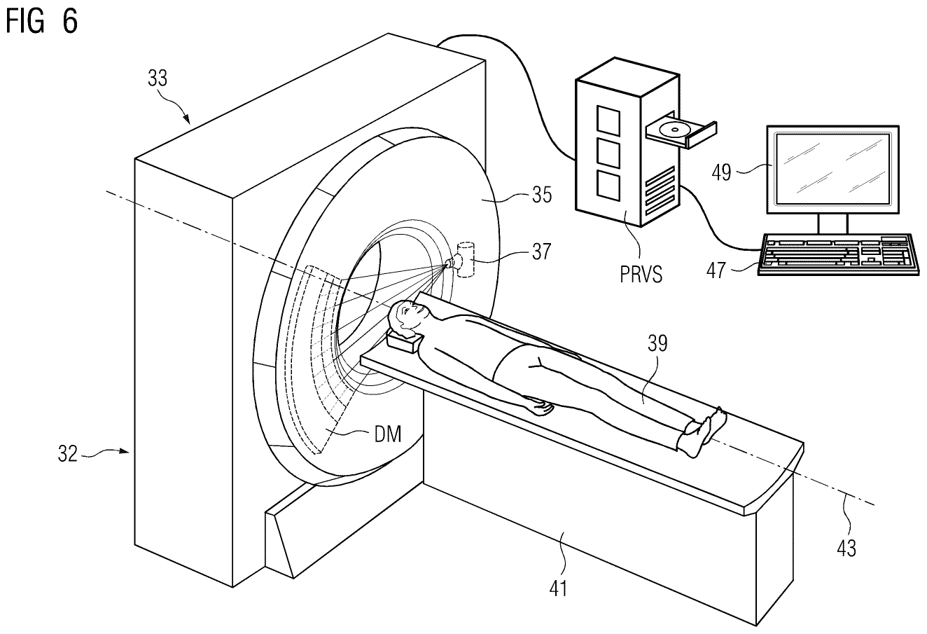

FIG. 6 shows a schematic representation of an advantageous embodiment of a proposed CT device 33 comprising an X-ray source 37, a CT detector module DM and a provider unit PRVS. The X-ray source 37 and the CT detector module DM can be arranged opposite one another. The X-ray source 37 can be designed to expose the CT detector module to X-ray radiation along a direction of incidence of the X-rays. The CT device 33 can also comprise a gantry 33 having a rotor 35. The X-ray source 37 and the CT detector module DM can be located on the rotor 35 in a defined arrangement, in particular integrated in the rotor 35 or attached to the rotor 35. The rotor 35 can be mounted to rotate about a rotational axis 43. The object 39 under examination to be imaged can be positioned on a patient couch 41 and be movable along the rotational axis 43 through the gantry 33. The provider unit PRVS can be used to control the CT device 32 and to compute sectional images or volumetric images of the object 39 under examination. An input device and/or input means 47, for example a keyboard, and an output apparatus 49, for example a screen and/or display, can be connected, in particular coupled for signal communication, to the provider unit PRVS. The input device 47 can advantageously be integrated in the output apparatus 49, for example given an, in particular resistive and/or capacitive, input display.

The schematic representations contained in the described figures are not shown to scale in any way and do not depict relative sizes.

Finally, it should be reiterated that the above methods described in detail and the presented apparatuses are merely exemplary embodiments, which can be modified by a person skilled in the art in many ways without departing from the scope of the present invention. In addition, the use of the indefinite article “a” or “an” does not rule out the possibility of there also being more than one of the features concerned. Likewise, the terms “unit” and “element” do not exclude the possibility that the components in question consist of a plurality of interacting sub-components, which may also be spatially distributed if applicable.

In the context of the present application, the expression “based on” can be understood in particular in the sense of the expression “using”. In particular, any wording, according to which a first feature is produced (or obtained, defined) on the basis of a second feature, does not exclude the possibility that the first feature is produced (or obtained, defined) on the basis of a third feature.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A cooling apparatus to dissipate heat from an X-ray converter element, the cooling apparatus comprising:
a plurality of cooling elements arranged in a row along a first direction, wherein
each of the plurality of cooling elements has a metallic material that has a plurality of continuous tunnel-shaped cavities along the first direction, the plurality of continuous tunnel-shaped cavities configured for through-flow by a fluid along the first direction,
the plurality of cooling elements are configured to make surface-to-surface contact with a corresponding thermal contact surface of the X-ray converter element, and
the plurality of cooling elements are arranged in the row such that respective tunnel-shaped cavities overlap at least partially at interfaces between adjacent cooling elements.

2. The cooling apparatus as claimed in claim 1, wherein each of the plurality of cooling elements has at least one of a recess or a protrusion on an external face, away from the first direction, the at least one of the recess or the protrusion configured to make surface-to-surface contact with the corresponding thermal contact surface of the X-ray converter element.

3. The cooling apparatus as claimed in claim 1, wherein
at least one of the plurality of cooling elements includes a plurality of cooling fins as part of the metallic material, the plurality of cooling fins being mutually spaced apart in a stacked arrangement,
a stacking direction of the plurality of cooling fins differs from the first direction, and
voids formed between the plurality of cooling fins form the plurality of continuous tunnel-shaped cavities.

4. The cooling apparatus as claimed in claim 3, wherein the stacking direction is perpendicular to the first direction.

5. The cooling apparatus as claimed in claim 1, wherein the plurality of continuous tunnel-shaped cavities are arranged equidistantly perpendicular to the first direction in a cross-sectional surface of an associated cooling element.

6. The cooling apparatus as claimed in claim 1, wherein the plurality of continuous tunnel-shaped cavities are spaced at different distances from one another perpendicular to the first direction in a cross-sectional surface of an associated cooling element.

7. The cooling apparatus as claimed in claim 1, wherein at least one of the plurality of cooling elements is configured as a single piece.

8. The cooling apparatus as claimed in claim 7, wherein the at least one of the plurality of cooling elements is produced via an additive manufacturing technique.

9. The cooling apparatus as claimed in claim 7, wherein the at least one of the plurality of cooling elements is produced via a subtractive manufacturing technique.

10. The cooling apparatus as claimed in claim 1, wherein different ones of the plurality of cooling elements in the row have at least one of a different cavity volume, a different cavity density, a different number of cavities, or a different cavity surface.

11. The cooling apparatus as claimed in claim 10, wherein, parallel to the first direction, the different ones of the plurality of cooling elements in the row have at least one of a decreasing cavity volume, a decreasing cavity density, an increasing number of cavities, or an increasing cavity surface.

12. The cooling apparatus as claimed in claim 11, wherein, parallel to the first direction, the plurality of cooling elements in the row have an increasing number of cooling fins.

13. The cooling apparatus as claimed in claim 1, wherein the plurality of cooling elements have a closed profile away from the first direction, so that the plurality of continuous tunnel-shaped cavities in the row of the plurality of cooling elements form a closed cooling duct along the first direction.

14. A CT detector module comprising:
the cooling apparatus as claimed in claim 1; and
at least one X-ray converter element including an X-ray detector layer on a top face and a thermal contact surface on a bottom face, wherein
in an operating state of the CT detector module, the at least one X-ray converter element is in heat-conducting contact with the cooling apparatus via the thermal contact surface.

15. The CT detector module as claimed in claim 14, wherein, in the operating state of the CT detector module, the at least one X-ray converter element is in heat-conducting contact with each of the plurality of cooling elements of the cooling apparatus via a corresponding thermal contact surface.

16. The CT detector module as claimed in claim 15, wherein
in the operating state of the CT detector module, the at least one X-ray converter element is in heat-conducting contact with each of the plurality of cooling elements of the cooling apparatus via a corresponding thermal contact surface by a thermally conductive medium, and
the thermally conductive media have different thermal conductivities.

17. A CT device comprising:
at least one CT detector module as claimed in claim 14.

18. The cooling apparatus as claimed in claim 2, wherein
at least one of the plurality of cooling elements includes a plurality of cooling fins as part of the metallic material, the plurality of cooling fins being mutually spaced apart in a stacked arrangement,
a stacking direction of the plurality of cooling fins differs from the first direction, and
voids formed between the plurality of cooling fins form the plurality of continuous tunnel-shaped cavities.

19. The cooling apparatus as claimed in claim 18, wherein the stacking direction is perpendicular to the first direction.

20. The cooling apparatus as claimed in claim 18, wherein the plurality of continuous tunnel-shaped cavities are arranged equidistantly perpendicular to the first direction in a cross-sectional surface of an associated cooling element.
